(19) United States
(12) Patent Application Publication
(10) Pub. No.: US 2018/0160890 A1
(43) Pub. Date: Jun. 14, 2018

(54) PREHEATING DEVICE FOR ENDOSCOPY

(71) Applicant: TEN COMFORT CORPORATION, New Taipei City (TW)

(72) Inventors: WEI-HSUN WANG, New Taipei City (TW); I-TING WANG, New Taipei City (TW); CHING-CHUAN WANG, New Taipei City (TW)

(21) Appl. No.: 15/840,252

(22) Filed: Dec. 13, 2017

(30) Foreign Application Priority Data
Dec. 14, 2016 (TW) 105219150

(51) Int. Cl. A61B 1/12

(52) U.S. Cl. CPC A61B 1/128

(57) ABSTRACT

A preheating device for endoscopy includes a flexible bag, a plurality of first welded joint, and a mark member. The flexible bag is disposed with a saturated sodium acetate solution and a trigger. The flexible bag includes a closed circumference around the outer periphery thereof. The closed circumference includes a longer side and a shorter side next to each other. The plurality of the first welded joint is arranged into a folding line along a longitudinal direction, wherein every two adjacent first welded joints of the folding line forms a flowing space. The folding line is substantially parallel to the longer side. The first welded joint nearest to the shorter side is the last first welded joint, and the mark member is disposed on the flexible bag between the last first welded joint and the shorter side. The mark member is used for indicating a placement of an endoscopy.

### CROSS-REFERENCE TO RELATED APPLICATION

This non-provisional application claims priority under 35 U.S.C. § 119(a) to Patent Application No. 105219150 filed in Taiwan, R.O.C. on Dec. 14, 2016, the entire contents of which are hereby incorporated by reference.

### BACKGROUND

### Technical Field

The present invention is related to a preheating device, especially to a preheating device for endoscopy.

### Related Art

During the medical examination or treatment, it is very common to use endoscopy. The endoscopy may be put inside of a human body for organ-inspection or surgery. In general, the endoscopy may be preheated before medical examination or treatment, so as not only to achieve the sterilized effect, but also to improve patient comfort during the medical examination or treatment.

A traditional method uses a hot pack to cover the outside of the endoscopy for preheating. In general, the hot pack stores with a saturated sodium acetate solution; and the saturated sodium acetate solution may cause an exothermic reaction to generate the heat. However, the traditional hot pack is formed with multiple enclosed spaces divided by the melted lines. Consequently, the circulation of the whole hot pack is not good. In some spaces, the saturated sodium acetate solution may start the exothermic reaction, while the reaction does not start in other spaces. This may cause a circulation problem. As a result, the preheating effect of the endoscopy is not good. In addition, when folding the hot pack for covering the outside of the endoscopy, the melted lines of the hot pack may not contact the surface of the endoscopy, due to depression. Consequently, the traditional hot pack covers the endoscopy incompletely, so the preheating effect is not good.

### SUMMARY

In order to solve aforementioned problems, one embodiment of the present disclosure provides a preheating device for endoscopy comprising a flexible bag, a plurality of first welded joints, and a mark member. The flexible bag is disposed with a saturated sodium acetate solution and a trigger. The flexible bag comprises a closed circumference around the outer periphery thereof. The closed circumference comprises a longer side and a shorter side next to each other. The plurality of the first welded joint is arranged into a folding line along a longitudinal direction, wherein every two adjacent first welded joints of the folding line focus a flowing space. The folding line is substantially parallel to the longer side. The first welded joint nearest to the shorter side is the last first welded joint, and the mark member is disposed on the flexible bag between the last first welded joint and the shorter side. The mark member is used for indicating a placement of an endoscopy.

In an embodiment of the present disclosure, the folding line of the preheating device is formed by the first welded joints arranged along a longitudinal direction, and every two adjacent first welded joints of the folding line forms the flowing space. When the trigger is triggered for chemical reaction, it releases heat. The heat is transmitted to every part of the preheating device. The endoscopy may be covered completely and preheated by the preheating device without protruding out of the flexible bag.

### BRIEF DESCRIPTION OF THE DRAWINGS

FIG. 1 is a schematic diagram showing the first embodiment of a preheating device for endoscopy of the instant disclosure;

FIG. 2 is a part enlarging view of the first embodiment of a preheating device for endoscopy of the instant disclosure;

FIG. 3 and FIG. 4 show the first embodiment of a preheating device for endoscopy of the instant disclosure in use (1) and (2) respectively;

FIG. 5 is a sectional view of FIG. 4;

FIG. 6 is a schematic diagram showing the second embodiment of a preheating device for endoscopy of the instant disclosure;

FIG. 7 is a schematic diagram showing the third embodiment of a preheating device for endoscopy of the instant disclosure;

FIG. 8 is a schematic diagram showing the forth embodiment of a preheating device for endoscopy of the instant disclosure;

FIG. 9 is a schematic diagram showing the fifth embodiment of a preheating device for endoscopy of the instant disclosure;

FIG. 10 is a schematic diagram showing the sixth embodiment of a preheating device for endoscopy of the instant disclosure; and

FIG. 11 is a schematic diagram showing the seventh embodiment of a preheating device for endoscopy of the instant disclosure.

### DETAILED DESCRIPTION

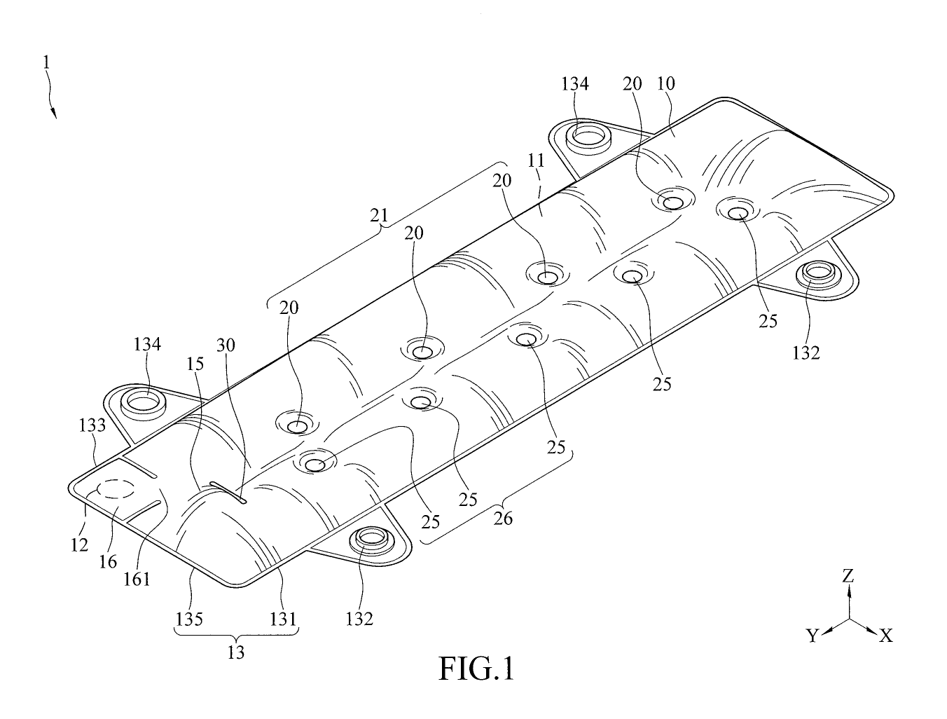

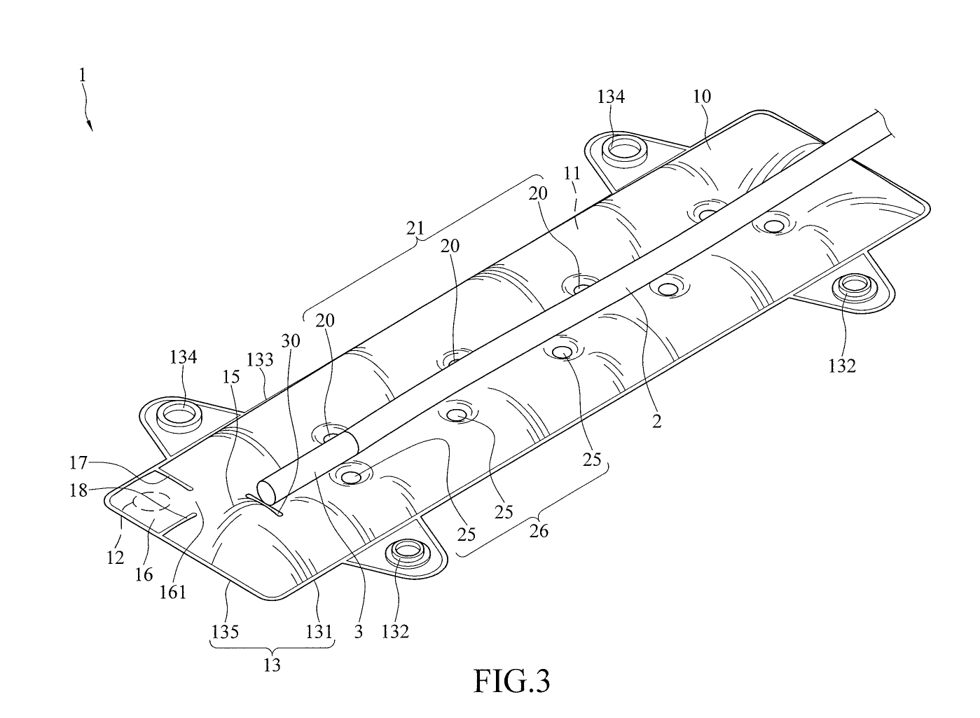

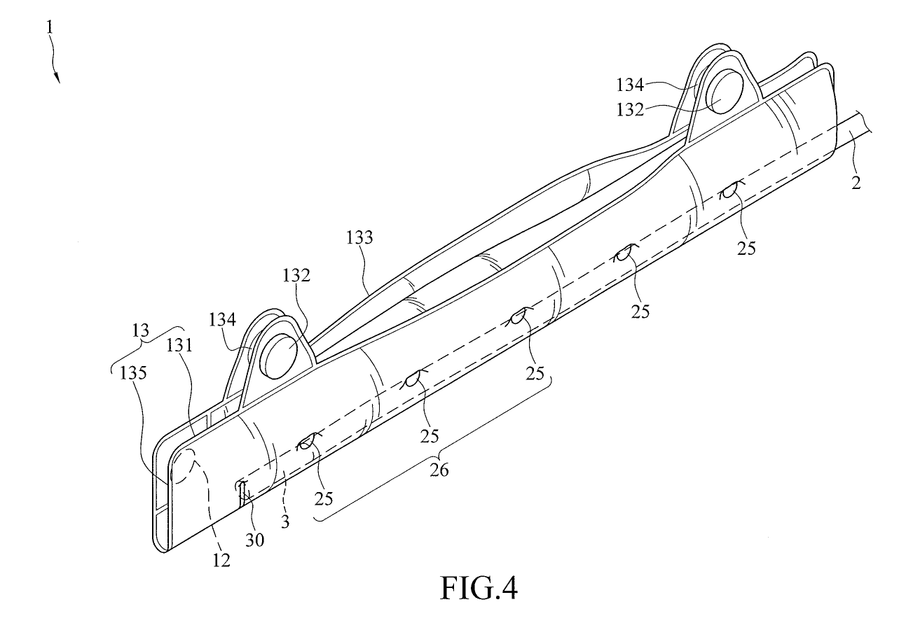

Please refer to FIG. 1, which is a schematic diagram showing the first embodiment of a preheating device for endoscopy of the instant disclosure. In this embodiment the preheating device for endoscopy 1 includes a flexible bag 10, a plurality of first welded joint 20, and a mark member 30. As shown in FIG. 3 and FIG. 4, the preheating device for endoscopy 1 covers on the endoscopy 2 for preheating the endoscopy 2.

As shown in FIG. 1, for example, the flexible bag 10 is rectangle in shape, which is not used to limit the present application, and the shape of the flexible bag 10 can be designed in other shape according to the demand of actual use. The flexible bag 10 includes a closed circumference 13 around thereof. For example, the closed circumference 13 may be formed by electric, high frequency, or ultrasonic high-heat welding the circumference of the flexible bag 10. Accordingly, the flexible bag 10 is formed as a closed bag, and the closed circumference 13 includes a longer side 131 and a shorter side 135 adjacent to each other. In addition, the flexible bag 10 is made by plastic polymer materials, which is flexible. For example, the flexible bag 10 may be made by polyvinyl chloride (PVC), polypropylene (PP), polycarbonate (PC), or other materials.

As shown in FIG. 1, the flexible bag 10 stores the saturated sodium acetate solution 11 within. The flexible bag 10 includes a trigger 12 contacting with the saturated sodium acetate solution 11 to start a chemical reaction in which the saturated sodium acetate solution 11 changes into a crystalline state from a saturation state. In this embodiment, the trigger 12 is a metal plate (such as an iron plate or a copper plate), and the user may bend or fold the metal plate to trigger the chemical reaction of the saturated sodium acetate solution 11. During the reaction from the saturation state into the crystalline state, the saturated sodium acetate solution 11 releases heat. The chemical reaction is: CH₃COO⁻_{(aq)}+Na⁺_{(aq)}+3H₂O_{(aq)}→CH₃COONa.3H₂O+heat. In one embodiment, the flexible bag 10 may be transparent or translucent so the user can identify the location of the trigger 12. In some other embodiment, the trigger 12 may be a vibrator located inside of the flexible bag 10. The chemical reaction of the saturated sodium acetate solution 11 is triggered by vibrating, which is not shown in the FIGS.

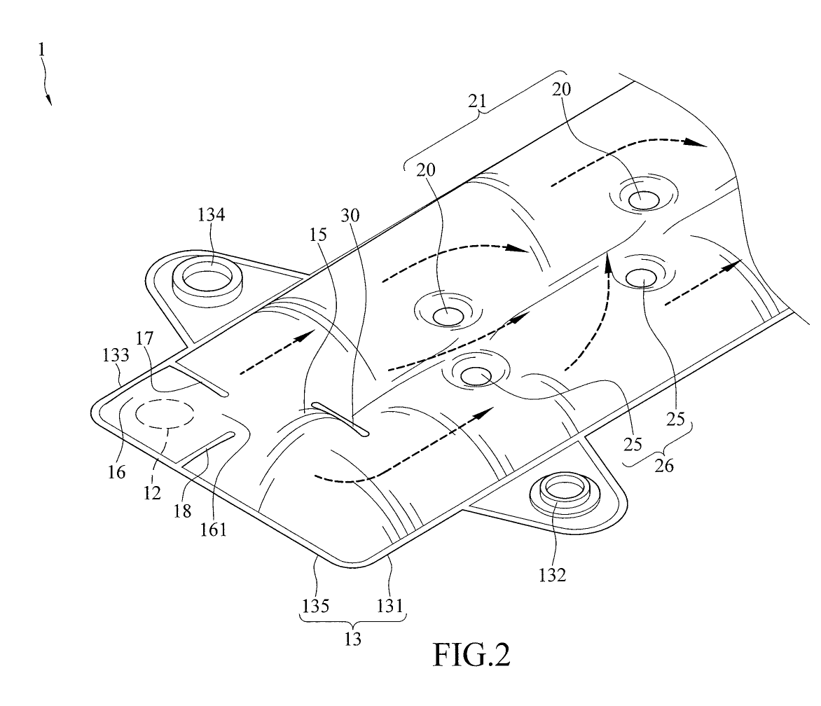

As shown in FIG. 1, in this embodiment the first welded joints 20 may be welded and formed on the flexible bag 10 by electric, high frequency, or ultrasonic high-heat. The first welded joints 20 are arranged along the longitudinal direction (i.e. Y-axis direction), and aligned in a folding line 21 with intervals. On the folding line 21, spaces between every two adjacent first welded joints 20 increase fluidiability, so the circulation inside the flexible bag 10 is increased. In other words, as shown in FIG. 2, the saturated sodium acetate solution 11 may easily flow between every two adjacent first welded joints 20 of the folding line 21 without obstruction. Additionally, the folding line 21 separates the flexible bag 10 into two parts (i.e. left part and right part). Every two adjacent first welded joints 20 has a predetermined interval, so the saturated sodium acetate solution 11 may easily flow from the left part of the flexible bag 10 through the intervals between the first welded joints 20 to the right part of the flexible bag 10, and vice versa. Accordingly, the inside of the flexible bag 10 may have good circulation. In some other embodiment, the intervals between the first welded joints 20 may vary depending on different designs. When the intervals between the first welded joints 20 are larger, the saturated sodium acetate solution 11 flows easier. In contrast, when the intervals between the first welded joints 20 are smaller, the saturated sodium acetate solution 11 flows more slowly.

As shown in FIG. 1, in this embodiment, the preheating device for endoscopy 1 includes two folding lines 21, 26, wherein the folding line 26 is formed by a plurality of second welded joint 25. The second welded joints 25 are also arranged along the longitudinal direction (i.e. Y-axis direction), and aligned in a folding line 21 with intervals. Accordingly, spaces between every two adjacent second welded joints 25 also increase fluidiability. The two folding lines 21, 26 are substantially parallel to the longer side 131 of the flexible bag 10 for folding and covering the endoscopy 2, as shown in FIG. 4. In addition, the two folding lines 21, 26 are substantially parallel to each other, and the welded joints 20 and the welded joints 25 are staggered, so the saturated sodium acetate solution 11 may flow easily between the two folding lines 21, 26. In some other embodiment, the preheating device for endoscopy 1 may include only one folding line, which is not shown in the FIGS.

As shown in FIG. 1, in this embodiment the first welded joint 20 next to the shorter side 135 is the last (first), welded joint of the folding line 21, and the second welded joint 25 next to the shorter side 135 is the last (second), welded joint. The mark member 30 is arranged on the flexible bag 10. The mark member 30 is located on between the last (first), welded joint and the shorter side 135 and located between the (second), welded joint and the shorter side 135. In other words, in comparison with the two last joints of the two folding lines 21, 26, the mark member 30 is closer to the shorter side 135. Additionally, there is a predetermined distance between the mark member 30 and the shorter side 135 for indicating the endoscopy 2. For example, if the flexible bag 10 is 35 cm long, the predetermined distance between the mark member 30 and the shorter side 135 may be 2 cm, 3 cm, 4 cm, or 5 cm.

As shown in FIG. 1 and FIG. 2, in this embodiment the mark member 30 may be a welded line formed on the flexible bag 10 by, for example, electric, high frequency, or ultrasonic high-heat. In one embodiment, the mark member 30 is substantially parallel to the shorter side 135 of the closed circumference 13 and perpendicular to the folding line 21. However, the figures are not used to limit the present application. In this embodiment the closed circumference 13 includes an opposite side 133 that is parallel to the longer side 131. The distance from a central point of the mark member 30 to the longer side 131 and the distance from the central point of the mark member 30 to the opposite side 133 are the same. The mark member 30 is shorter than the shorter side 135. For example, the length of the mark member 30 may be ⅓, ¼, or ⅕ of the shorter side 135. When the user places the endoscopy 2 according to the indication of the mark member 30, the endoscopy 2 may be located in the center area of the flexible bag 10, which is between the two folding lines 21, 26. In addition, when the mark member 30 is a welded line, a blocker 15 may be protruded between the mark member 30 and the shorter side 135 of the closed circumference 13.

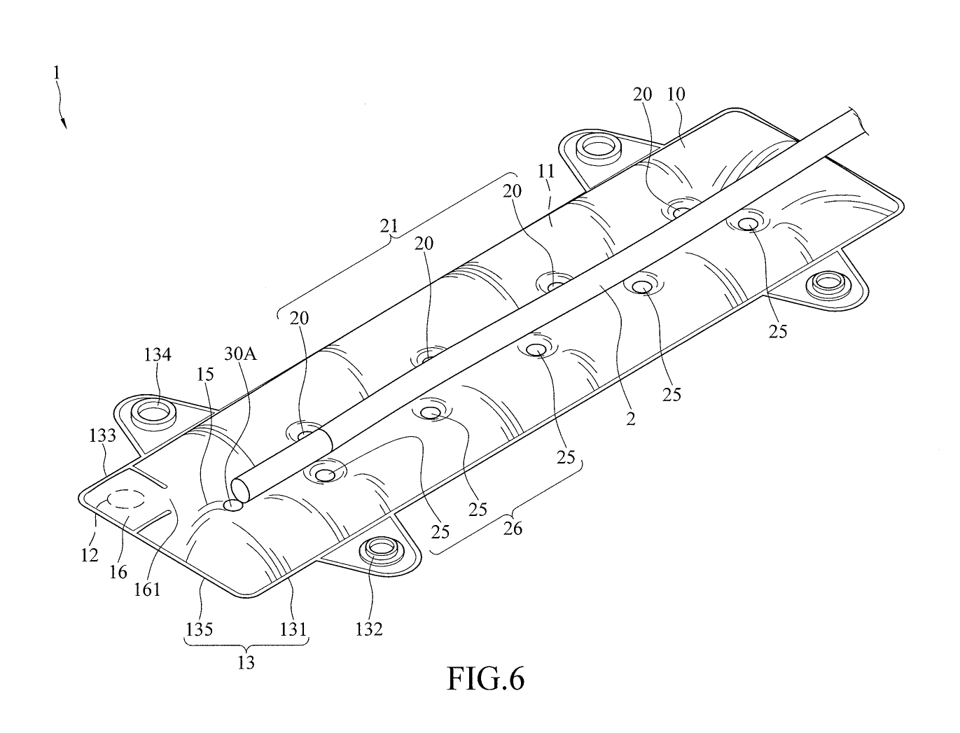

FIG. 6 is a schematic diagram showing a second embodiment of a preheating device for endoscopy of the instant disclosure. The difference between the first and the second embodiment is that the mark member 30A is a first welded joint. The distance from the mark member 30A to the longer side 131 and the distance from the mark member 30A to the opposite side 133 are the same. Similarly, the blocker 15 may also be protruded between the mark member 30 and the shorter side 135 of the closed circumference 13. When the user places the endoscopy 2 according to the indication of the mark member 30A, the endoscopy 2 may be located in the center area of the flexible bag 10. In some other embodiment, the mark member 30A and the first welded joints 20 may be on the same line; alternatively, the mark member 30A and the second welded joints 25 may be on the same line, which are not shown in the FIGS.

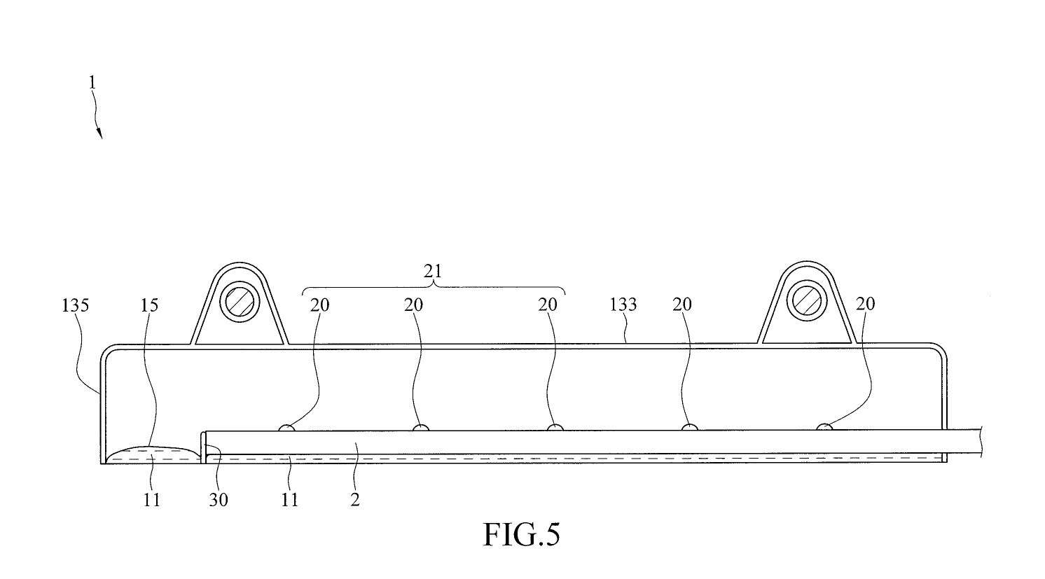

The following paragraphs will describe a preheating procedure of one embodiment of the disclosure. Accompanying FIG. 1, the user may flatten the preheating device for endoscopy 1 on a plane. Next, as shown in FIG. 3, the user may put the camera 3 of the endoscopy 2 on the mark member 30 so as to allow the endoscopy 2 being located on the center area of the flexible bag 10 and to avoid the camera 3 of the endoscopy 2 closing to the shorter side 135 of the closed circumference 13. Accordingly, the endoscopy 2 may be kept without extending out of the shorter side 135 of the closed circumference 13, and the camera 3 of the endoscopy 2 may be preheated properly. As shown in FIG. 4, when the user places the endoscopy 2 as indicated by the mark member 30, the flexible bag 10 is folded along with the two folding lines 21, 26 to cover the endoscopy 2. Next, the user may fold the trigger 12 (for example a metal plate), to trigger the chemical reaction of the saturated sodium acetate solution 11. During the reaction from the saturation state into the crystalline state, the saturated sodium acetate solution 11 releases heat. The heat may reach the endoscopy 2 so as to preheat the endoscopy 2. Please see FIG. 2. On the folding line 21, flowing spaces between every two adjacent first welded joints 20 increase the fluidiability. Similarly, flowing spaces between every two adjacent second welded joints 25 also increase the fluidiability. Accordingly, the inside circulation of the flexible bag 10 may be improved greatly. Consequently, the heat may be transmitted to the preheating device 1 quickly without the obstruction of folding lines 21, 26, so as to preheat the whole preheating device 1. In addition, please see FIG. 3 and FIG. 5. In this embodiment the blocker 15 protruded between the mark member 30 and the shorter side 135 of the closed circumference 13 is used to prevent the movement of the endoscopy 2 and prevent the camera 3 extending out of the shorter side 135 of the closed circumference 13, so as preheat the endoscopy 2. Additionally, the welded joints 20 on the folding line 21 and the welded joints 25 on the folding line 26 are staggered. That is, every two adjacent first welded joints 20 on the folding line 21 are separated without being welded together. Similarly, the every two adjacent second welded joint 25 on the folding line 26 are separated without being welded together. When the flexible bag 10 is folded to cover the endoscopy 2, protruding areas between every two adjacent first welded joints 20 and between every two adjacent second welded joints 25 may contact with the surface of the endoscopy 2. Accordingly, the contact surface of endoscopy 2 is largely increased, and the preheating effect of the endoscopy 2 is greatly improved.

As shown in FIG. 3, in one embodiment, when the user put the endoscopy 2 indicated by the mark member 30, they may first fold the trigger 12 to trigger the chemical reaction of the saturated sodium acetate solution 11. Before the reaction from the saturation state into the crystalline state is completed, the user may fold the flexible bag 10 along with the two folding lines 21, 26 to cover the endoscopy 2 for preheating.

As shown in FIG. 4 and FIG. 5, in another embodiment, the user may fold the flexible bag 10 first, and then put the endoscopy 2 into folded area of the flexible bag 10 from the opposite side of the shorter side 135. The blocker 15 between the mark member 30 and the shorter side 135 of the closed circumference 13 may limit the endoscopy 2 being placed on a proper location. In another words, when the user put the endoscopy 2 into the folded area of the flexible bag 10 and stop by the blocker 15, the endoscopy 2 may be placed on a predetermined place. Even when the endoscopy 2 is forced to move, it may stop by the blocker 15. Accordingly, it may prevent the camera 3 of the endoscopy 2 protruding out of the shorter side 135 of the closed circumference 13. Finally, the user folds the trigger 12 to trigger the chemical reaction of the saturated sodium acetate solution 11. During the reaction from the saturation state into the crystalline state, the saturated sodium acetate solution 11 releases heat so as to preheat the endoscopy 2.

As shown in FIG. 3, in this embodiment the longer side 131 of the closed circumference 13 include two first connecting members 132 (male buckles), disposed on two side of the flexible bag 10, respectively. The opposite side 133 of the closed circumference 13 include two second connecting members 134 (female buckles), corresponding to the first connecting member 132. As shown in FIG. 4, when the flexible bag 10 is folded along the folding lines 21, 26, the first connecting members 132 and the second connecting members 134 are used to connect with each other, so as to keep the flexible bag 10 in a folding state. However, the Figures are not used to limit the present application. In some other embodiment, the first connecting member(s) 132 and the second connecting member(s) 134 may be Velcro, straps, or the like with male and female members corresponding to each other. In addition, longer side 131 of the closed circumference 13 may include only one first connecting member 132, and the opposite side 133 of the closed circumference 13 may include only one second connecting member 134 corresponding to the first connecting member 132.

In one embodiment, the trigger 12 may be disposed near the mark member 30. As shown in FIG. 1 and FIG. 2, the flexible bag 10 includes a room 16 near the mark member 30. In this embodiment the mark member 30 is near the corner of the shorter side 135 of the closed circumference 13. In addition, the room 16 includes a connecting thoroughfare 161 for the saturated sodium acetate solution 11 flowing to the room 16. In this embodiment the trigger 12 is disposed within the room 16. When the user folds the trigger 12 to trigger the chemical reaction of the saturated sodium acetate solution 11. An area near the shorter side 135 of the flexible bag 10 may first release heat, so the camera 3 of the endoscopy 2 may first be heated. The whole preheating procedure for the endoscopy 2 may be faster.

As shown in FIG. 1 and FIG. 2, in this embodiment the preheating device for endoscopy 1 includes a first welded part 17 and a second welded part 18. The first welded part 17 is a welded line extending from the opposite side 133 of the closed circumference 13 and parallel to the shorter side 135. The second welded part 18 is a welded line extending from the shorter side 135 of the closed circumference 13 and parallel to the opposite side 133. The room 16 is substantially formed by the first welded part 17, the second welded part 18, the opposite side 133, and the shorter side 135, wherein the first welded part 17 and the second welded part 18 do not extend to contact each other and form the connecting thoroughfare 161. In some embodiments, the first welded part 17 and the second welded part 18 may be formed by one first welded joint or multiple first welded joints, which is not shown in the figures.

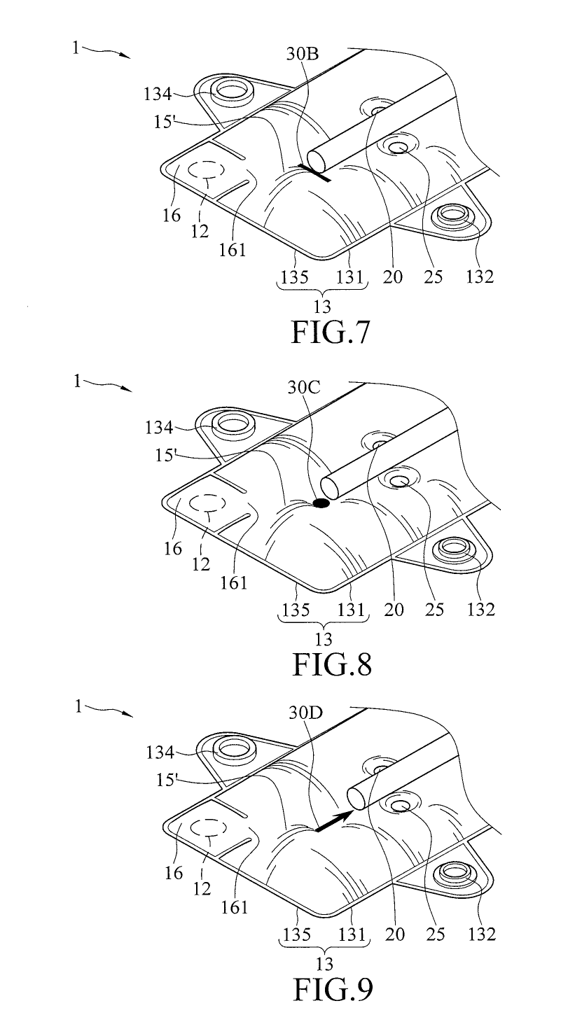

In some embodiment, the mark member 30 of the preheating device for endoscopy 1 may also be a mark line, a mark dot, a symbol, a mark pattern, or a word, such as a punctuation, a mark, or a word formed by printing on the inner or outer surface of the flexible bag 10, which is not formed by welded. FIG. 7 is a schematic diagram showing the third embodiment of a preheating device for endoscopy of the instant disclosure. In this embodiment the mark member 30B is a mark line substantially parallel to the shorter side 135 of the closed circumference 13. FIG. 8 is a schematic diagram showing the forth embodiment of a preheating device for endoscopy of the instant disclosure. In this embodiment the mark member 30C is a mark point. The distance between the mark point and longer side 131 is same as the distance between the mark point and the opposite side 133. The mark point may locate at other position. For example, the mark point may be on the same line of the first welded joints 20 or the second welded joints 25. FIG. 9 is a schematic diagram showing the fifth embodiment of a preheating device for endoscopy of the instant disclosure. In this embodiment the mark member 30D is a mark pattern (an arrow in this embodiment, however it may be other pattern or word).

As shown in FIG. 7, in this embodiment a blocker 15′ is also protruded between the mark member 30B and the shorter side 135 of the closed circumference 13. The blocker 15′ may be formed when producing the flexible bag 10. For example, the blocker 15′ may be formed between the mark member 30B and the shorter side 135 by thermosetting. Please see FIG. 8 and FIG. 9. The blocker 15′ in the embodiment of FIG. 8 and FIG. 9 may also be formed when producing flexible bag 10 using the same method.

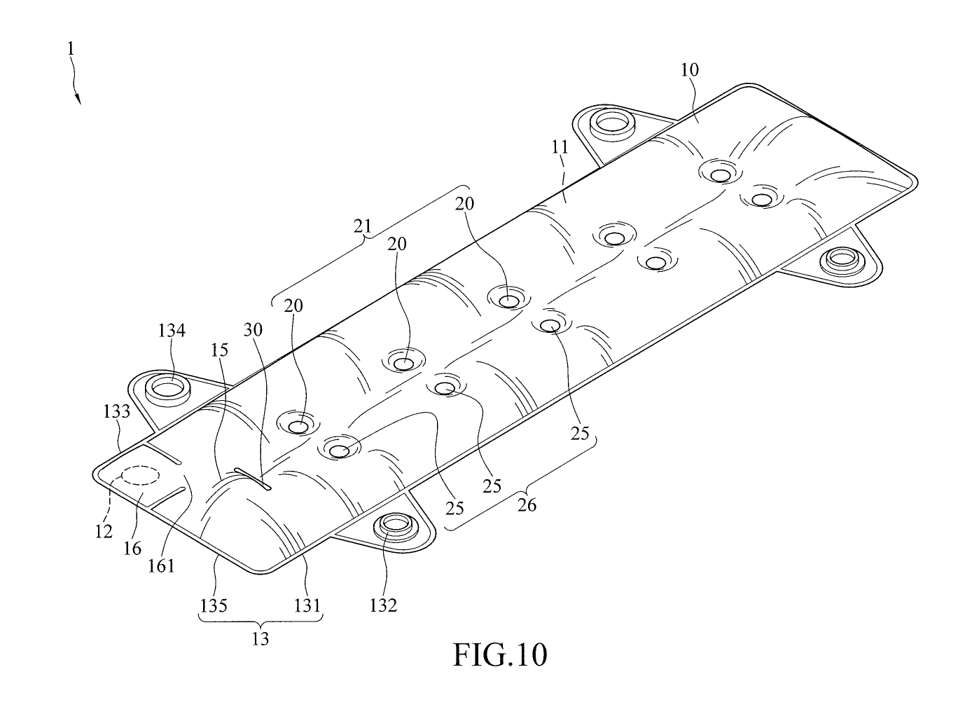

In one embodiment, the arrangement of the first welded joints 20 of the folding line 21 and the arrangement of the second welded joints 25 of the folding line 26 may be different. For example, as shown in FIG. 1, the number of the first welded joints 20 of the folding line 21 is 4, and the number of the second welded joints 25 of the folding line 26 is 5. Consequently, the first welded joints 20 and the second welded joints 25 are staggered. The distance between every two welded joints 20 is different from the distance between every two second welded joints 25. FIG. 10 is a schematic diagram showing the sixth embodiment of a preheating device for endoscopy of the instant disclosure. In this embodiment, the number of the first welded joints 20 of the folding line 21 and the number of the second welded joints 25 of the folding line 26 both are 5. The first welded joints 20 and the second welded joints 25 are aligned with each other, respectively. The distance between every two welded joints 20 is same as the distance between every two second welded joints 25, so the fluidability between welded joints may be the same inside of the flexible bag 10 and the heat may be released more equally.

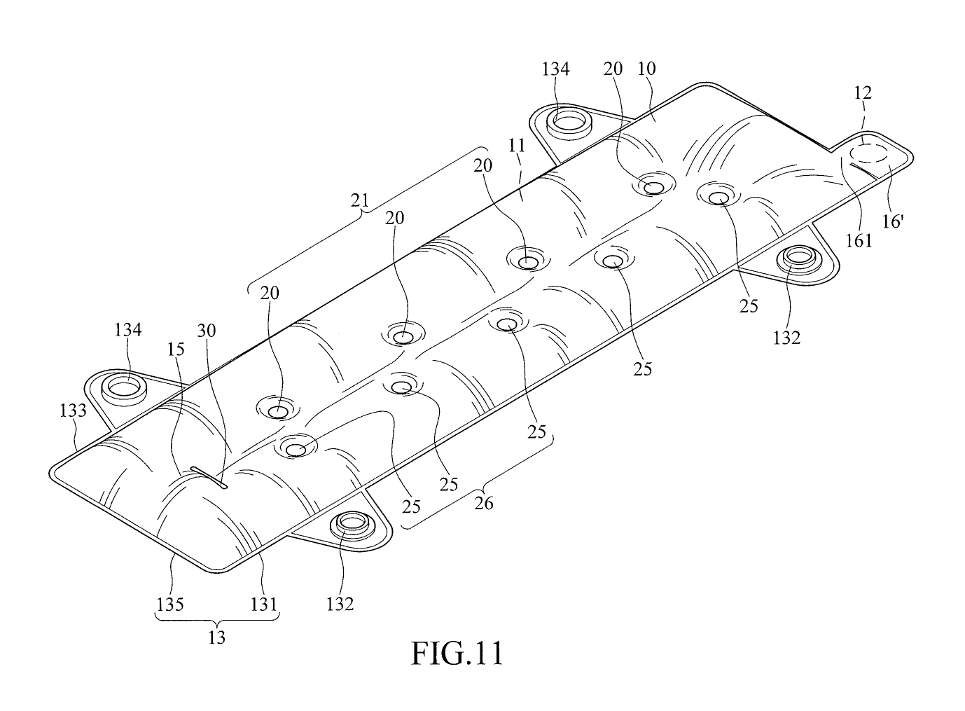

FIG. 11 is a schematic diagram showing the seventh embodiment of a preheating device for endoscopy of the instant disclosure. In this embodiment the room 16′ is protruding out of the closed circumference 13. When the user folds the flexible bag 10, the room 16′ is protruded. The user may folds the trigger 12 more easily to trigger the chemical reaction of the saturated sodium acetate solution 11 for releasing heat. Additionally, in this embodiment the room 16′ is located at the side far away from the mark member 30 of the flexible bag 10. The figures are not used to limit the present application. The room 16′ may be located at any other side of the flexible bag 10.

In summary, according to the embodiments of the preheating device for endoscopy of present disclosure, the first welded joints arranged along the longitudinal direction forms the folding line. The flowing spaces between every two welded joints provide fluidability. When the trigger triggers the chemical reaction of the saturated sodium acetate solution to release the heat, it may be transmitted to the whole preheating device quickly. Consequently, when the flexible bag covers the endoscopy, it preheats the endoscopy. In addition, the mark member may be used to indicate the placing location of the endoscopy, so the endoscopy may be completely covered by the preheating device and stops the endoscopy protruding out of the preheating device.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A preheating device for endoscopy comprising:
a flexible bag comprising a closed circumference around the outer periphery thereof, wherein the closed circumference comprises a longer side and a shorter side next to each other, wherein the flexible bag is disposed with a saturated sodium acetate solution and a trigger;
a plurality of the first welded joint arranged into a folding line along a longitudinal direction, wherein every two adjacent first welded joints of the folding line forms a flowing space, wherein the folding line is substantially parallel to the longer side; and
a mark member disposed on the flexible bag, wherein the mark member is used for indicating a placement of an endoscopy.

2. The preheating device for endoscopy according to claim 1, wherein the first welded joint nearest to the shorter side is the last first welded joint, and the mark member is between the last first welded joint and the shorter side.

3. The preheating device for endoscopy according to claim 1, wherein the mark member may be a mark line, a mark dot, a mark symbol, a mark pattern, or a mark word.

4. The preheating device for endoscopy according to claim 1, wherein the mark member is a welded line.

5. The preheating device for endoscopy according to claim 4, wherein the welded line is substantially parallel to the shorter side.

6. The preheating device for endoscopy according to claim 1, wherein the mark member is a second welded joint.

7. The preheating device for endoscopy according to claim 1, wherein a blocker is protruded between the mark member and the shorter side.

8. The preheating device for endoscopy according to claim 2, wherein a blocker is protruded between the mark member and the shorter side.

9. The preheating device for endoscopy according to claim 3, wherein a blocker is protruded between the mark member and the shorter side.

10. The preheating device for endoscopy according to claim 4, wherein a blocker is protruded between the mark member and the shorter side.

11. The preheating device for endoscopy according to claim 5, wherein a blocker is protruded between the mark member and the shorter side.

12. The preheating device for endoscopy according to claim 6, wherein a blocker is protruded between the mark member and the shorter side.

13. The preheating device for endoscopy according to claim 1 further comprising a plurality of second welded joint arranged into an another folding line along a longitudinal direction, wherein every two adjacent second welded joints of the folding line forms an another flowing space, wherein the another folding line is substantially parallel to the folding line, and wherein the second welded joint nearest to the shorter side is the last second welded joint.

14. The preheating device for endoscopy according to claim 13, wherein the first welded joints and the second welded joints are staggered or aligned.

15. The preheating device for endoscopy according to claim 13, wherein a first distance between every two adjacent first welded joints is same as a second distance between every two adjacent second welded joints.

16. The preheating device for endoscopy according to claim 1, wherein the closed circumference comprises an opposite side corresponding to the longer side, wherein the longer side is disposed with at least a first connecting member and the opposite side is disposed with at least a second connecting member, and wherein the at least the first connecting member is corresponding to the at least the second connecting member for fixing the folding bag after being folded along the folding line.

17. The preheating device for endoscopy according to claim 1, wherein the trigger is disposed within the flexible bag near the mark member.

18. The preheating device for endoscopy according to claim 1, wherein the flexible bag further comprises a room for disposing the trigger, and wherein the room comprises a connecting thoroughfare.

19. The preheating device for endoscopy according to claim 18, wherein the room is protruding out of the closed circumference.

20. The preheating device for endoscopy according to claim 19, wherein the room is located at a side far from the mark member on the flexible bag.
